(19) United States
(12) Patent Application Publication
(10) Pub. No.: US 2007/0060807 A1
(43) Pub. Date: Mar. 15, 2007

(54) Detector

(75) Inventor: Yoshihiro Oishi, Ikoma-gun (JP)

Correspondence Address: BIRCH STEWART KOLASCH & BIRCH, PO BOX 747, FALLS CHURCH, VA, 22040-0747, US

(73) Assignee: Sharp Kabushiki Kaisha

(21) Appl. No.: 11/507,541

(22) Filed: Aug. 22, 2006

(30) Foreign Application Priority Data
Aug. 26, 2005 (JP) 2005-245972

(51) Int. Cl. A61B 5/00

(52) U.S. Cl. 600/322; 600/310

(57) ABSTRACT

A pair of light-receiving sections are placed at positions symmetric to a light-emitting axis of a light-emitting section on the inner circumferential surface in close contact with a finger, and each light-receiving section is composed of a plurality of light receiving regions so that by selecting a signal of the light receiving region which maximizes a reception signal, the propriety and the correction direction of a wearing position are displayed. According to the ring sensor, it becomes possible to enhance light-receiving efficiency of the light-receiving section and to increase a signal-noise ratio. It also becomes possible to facilitate wearing of the ring sensor at an optimum position.

### CROSS-REFERENCE TO RELATED APPLICATIONS

This nonprovisional application claims priority under 35 U.S.C. §119(a) on Patent Application No(s). 2005-245972 filed in Japan on Aug. 26, 2006, the entire contents of which are hereby incorporated by reference.

### BACKGROUND OF THE INVENTION

The present invention relates to a sensor for optically measuring biological information in a noninvasive manner.

Conventionally, ring sensors which have a light-emitting section and a light-receiving section provided on the inner circumferential surface of a ring have been used for optically detecting pulse waves.

It is known that among hemoglobin in the blood, oxyhemoglobin and reduced hemoglobin are different in light absorption and transmission characteristics depending on wavelengths of light. The oxyhemoglobin absorbs infrared light more than red light, whereas the reduced hemoglobin has an optical characteristic to absorb red light more than infrared light.

Therefore, by wearing the ring sensor on a finger and pressing the light-receiving section and the light-emitting section to the finger so that a specified pressure is applied in advance onto a blood vessel in a finger, the amount of light received by the light-receiving section decreases during a vasodilator period by pulses whereas the amount of light increases during a vasoconstriction period.

FIG. 7 is a view showing absorbance when light is transmitted through the human body. The absorbance consists of an AC component which is a pulse component attributed to arterial blood and a DC component which is a non-pulse component attributed to venous blood and tissue. Because of the AC component and DC component in the absorbance, the amount of light received by the light-receiving section also consists of an AC component and a DC component. The amount of light is analyzed so as to calculate pulse rates and oxygen saturation in blood.

More specifically, when light passes through blood vessels, the amount of light is subjected to periodical modulation by pulses, and therefore, measuring a period of amplitude change in a light reception signal outputted by the light-receiving section makes it possible to obtain a pulse rate. Moreover, since a ratio between red light and infrared light received changes by a ratio between oxyhemoglobin and reduced hemoglobin, analyzing a ratio of the respective reception signal intensity values makes it possible to obtain oxygen saturation in blood.

Such ring sensors include a so-called transmission type in which a light-receiving axis of the light-receiving section and a light-emitting axis of the light-emitting section face each other so as to align for receiving the light transmitted through blood vessels.

There is also a so-called reflection type in which the light-receiving axis and the light-emitting axis are placed so as to intersect with each other at a specified angle for receiving the light reflected or diffused by blood vessels as shown in JP 2002-224088 A.

In the case where the light-emitting axis and the light-receiving axis are placed so as to align in the transmission-type, proportion of the DC component in light reception signals is high. This is presumably because the proportion of a component of outgoing light from the light-emitting section that directly comes incident into the light-receiving section is higher than the proportion of a component of outgoing light from the light-emitting section that diffuses in tissue and comes incident into the light-receiving section. As for detection of pulse waves, it is achieved by the AC component in a light reception signal.

Consequently, since the proportion of the DC component is relatively larger than the proportion of the AC component in the light emitted from the light-emitting section in the transmission type, detection of pulse waves has a problem of a low signal-noise ratio.

In the reflection-type ring sensor as disclosed in JP 2002-224088 A, light-receiving efficiency is poor since reflected light or diffused light is received, and therefore it is necessary to increase the amount of light emitted from the light-emitting section or to enlarge a light-receiving area of the light-receiving section corresponding to the light-emitting section. Increasing the amount of light may cause low-temperature burn on the human body in long-time wearing.

In the case of enlarging the receiving area of a single light-receiving section, the probability of picking up disturbance light, which is not a measuring object, increases, and this causes the problem of a low signal-noise ratio.

Moreover, in detection of pulse waves, there is a problem of the low signal-noise ratio depending on wearing positions. Moreover, because of the ring shape, the ring sensor is noticeably displaced in the rotation direction by body motion. In this case, the AC component decreases, which causes the problem of the low signal-noise ratio.

Moreover, with a single light-receiving section, it is difficult to determine whether or not a wearing position is optimum and it is impossible to detect the direction in which a wearing position is displaced.

### SUMMARY OF THE INVENTION

An object of the present invention is to provide, in view of the problems, a detector which enhances light-receiving efficiency. Another object of the present invention is to provide a detector allowing stable detection of reception signals even with displacement of a wearing position. Still another object of the present invention is to provide a detector with high safety even in long-time wearing.

In order to achieve the above object, there is provided a detector, comprising:

a light-emitting section for radiating light; and

a light-receiving section for receiving light, the light-emitting section and the light-receiving section being formed on an inner circumferential surface of a finger ring-type ring,

wherein the light-receiving section is composed of at least a first light-receiving section and a second light-receiving section, and

wherein each light-receiving section is placed at a position symmetric to a light-emitting axis of the light-emitting section.

In one embodiment of the invention, the detector further comprises:

a comparing section for comparing first and second reception signals outputted from the first and second light-receiving sections;

a selecting section for selecting any one of the first reception signal, the second reception signal, and a sum of the first and second reception signals based on a comparison result in the comparing section; and

a signal processing section for calculating biological information from the reception signal selected in the selecting section.

In one embodiment of the invention, propriety of a wearing position is determined based on the comparison result.

In one embodiment of the invention, the first and second light-receiving sections have a plurality of light-receiving regions along the inner circumferential surface.

In one embodiment of the invention, the respective light-receiving sections have at least three light-receiving regions, and

wherein light-receiving regions each positioning in central portions of the respective light-receiving sections are placed at positions facing each other on the inner circumferential surface.

In one embodiment of the invention, the first and second reception signals outputted from the first and second light-receiving sections are signals having a maximum amplitude among reception signals of the respective light-receiving regions, and propriety of a wearing position is determined by a combination of the light-receiving regions corresponding to the first and second reception signals.

In one embodiment of the invention, a correction direction of the wearing position is determined based on the combination.

In one embodiment of the invention, the detector further comprises a signal processing section for calculating biological information from the reception signals selected based on the combination.

In one embodiment of the invention, the light-receiving axis of the light-receiving section and the light-emitting axis of the light-emitting section are in one plane.

In one embodiment of the invention, a light-emitting point of the light-emitting section and a light-receiving point of the light-receiving section are placed at different tracks on the inner circumferential surface.

In one embodiment of the invention, the light-emitting axis and the light-receiving axis are placed so as to intersect with each other.

In one embodiment of the invention, the first light-receiving section and the second light-receiving section are placed at positions facing each other on the inner circumferential surface.

In one embodiment of the invention, the detector further comprises an amplifier for amplifying a reception signal outputted from the light-receiving sections,

wherein an amplification factor of the amplifier is adjusted based on the reception signal inputted into the signal processing section.

In one embodiment of the invention, the biological information calculated from the reception signal by the signal processing section is a pulse or oxygen saturation in blood.

According to the structure of the present invention, it becomes possible to enhance the light-receiving efficiency of the detector. It also becomes possible to stably detect reception signals even with displacement of the wearing position. Moreover, the detector is harmless to the human body in long-time wearing.

That is, the detector has at least two or more light-receiving sections, and this makes it possible to enhance the light-receiving efficiency without the necessity of increasing the light emission amount in the light-emitting section.

Even when the wearing position is displaced by body motion, selection and combination of a plurality of reception signals makes it possible to suppress decrease in signal-noise ratio in the reception signals.

Moreover, since the light emission amount for each light-emitting section can be reduced, it becomes possible to decrease the possibility of causing low-temperature burn on the human body in long-time wearing.

Moreover, since the light-receiving area of each light-receiving section can be reduced, it becomes possible to reduce the probability of picking up any disturbance light, which is other than pulse wave signals. Therefore, the signal-noise ratio in the reception signals can be increased.

Moreover, the proportion of the AC component in a reception signal increases, and this can increase the signal-noise ratio in the reception signals.

Moreover, it becomes possible to determine whether or not the wearing position of the detector is optimum. In addition, it is possible to display the correction direction for an optimum wearing position, and therefore users can easily correct the wearing position.

### BRIEF DESCRIPTION OF THE DRAWINGS

The present invention will become more fully understood from the detailed description given hereinbelow and the accompanying drawings which are given by way of illustration only, and thus are not intended to limit the present invention, and wherein:

FIG. 1A is a perspective view showing a ring sensor in a worn state in an embodiment 1 of the present invention, FIG. 1B is a perspective view showing the ring sensor, FIG. 1C is a transverse sectional view showing the ring sensor, FIG. 1D is a view showing a directional pattern of a light-emitting section in the ring sensor, and FIG. 1E is a view showing a directional pattern of a light-receiving section in the ring sensor;

FIG. 2A is a block diagram showing the ring sensor, and FIG. 2B is a view showing the operation of a comparing section;

FIG. 3A is perspective view showing a ring sensor in an embodiment 2 of the present invention, FIG. 3B is a transverse sectional view showing the ring sensor, FIG. 3C is a cross sectional view taken along an arrow line A-A′ in FIG. 3B, and FIG. 3D is a cross sectional view taken along an arrow line B-B′ in FIG. 3B;

FIG. 4A is a perspective view showing a ring sensor in a worn state in an embodiment 3 of the present invention, and FIG. 4B is a transverse sectional view showing the ring sensor;

FIG. 5A is a block diagram showing the ring sensor in FIGS. 4A and 4B, and FIG. 5B is a view showing a matrix and the operation of a display section of the ring sensor;

FIG. 6 is a transverse sectional view showing a ring sensor in an embodiment 4; and

FIG. 7 is a view showing the absorbance when light is transmitted through the human body.

### DETAILED DESCRIPTION OF THE INVENTION

Hereinbelow, the embodiments of the detector in the present invention will be described with reference to the accompanying drawings.

It is to be noted that in the embodiments, the detector of the present invention is referred to as a ring sensor.

(Embodiment 1)

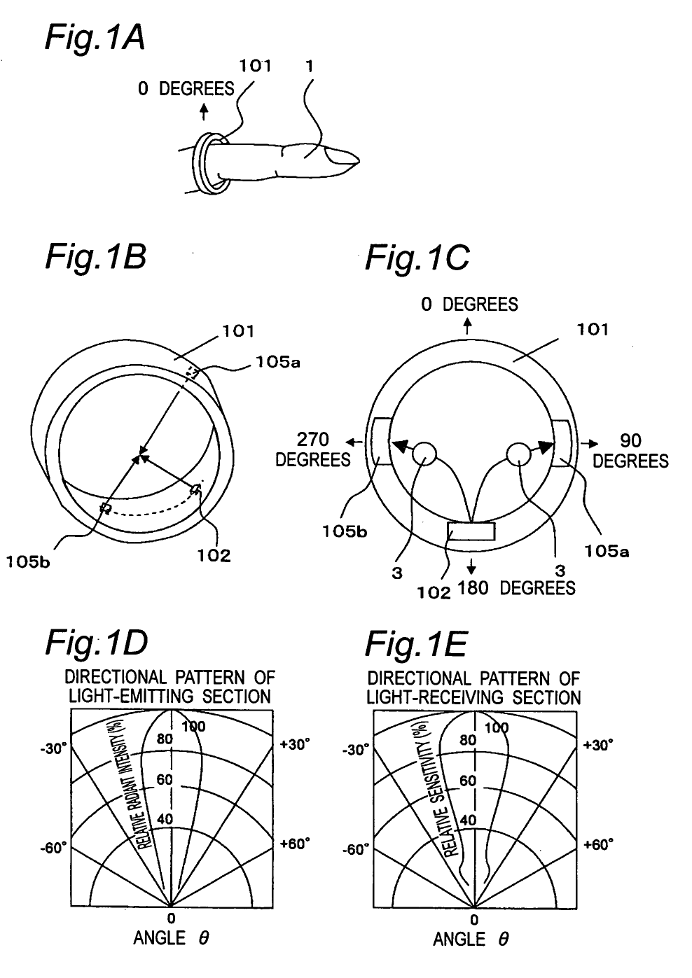

FIGS. 1A to 1E are views about a ring sensor in an embodiment 1.

As shown in FIG. 1A, a ring sensor 101, which is a finger ring-type ring, is worn on the base of a finger 1. A ring size is appropriately selected according to the size of a finger of each user so that the inner circumferential surface of the ring is constantly in close contact with the finger.

FIGS. 1B and 1C show the inner circumferential surface and the cross section of the ring sensor. On the inner circumferential surface, there are a light-emitting section 102 formed from a light-emitting diode, and first and second light-receiving sections 105a, 105b formed from photodiodes. The light-emitting section 102 has a light-emitting diode for emitting red light and a light-emitting diode for emitting infrared light.

FIG. 1D shows an example of the directional patterns of the light-emitting section and the light-receiving section. The light-emitting section 102 and the respective light-receiving sections 105 have maximum relative radiant intensity and maximum relative sensitivity in a direction perpendicular to a light-emitting face and a light-receiving face, and central axes of the intensity and sensitivity distribution carves are referred to as a light-emitting axis and a light-receiving axis.

In FIGS. 1B and 1C, the light-emitting section 102 is formed on the inner circumferential surface of the ring, and the first and second light-receiving sections 105a, 105b are formed at positions symmetric to the light-emitting axis of the light-emitting section 102.

More specifically, the light-emitting section 102, and the first and second light-receiving sections 105a, 105b are placed so that the first and second light-receiving sections 105a and 105b are placed at the positions facing each other on the inner circumferential surface of the ring sensor and that the light-emitting axis and the respective light-receiving axes are in one plane.

In other words, assuming that a portion in contact with the back of the finger be 0 degrees, the first and second light-receiving sections 105a, 105b are placed at the positions of 90 degrees and 270 degrees clockwise, while the light-emitting section 102 is placed at the position of 180 degrees.

The state that the back of the finger is aligned with the position of 0 degrees is regarded as a reference position. In order to allow easy visual confirmation of the sensor being worn at the reference position, an identification marking or protrusion should preferably be provided on the ring.

In the thus-structured ring sensor, light emitted from the light-emitting section 102 touches a blood vessel 3 of the finger 1 and reflects or diffuses before reaching the first and second light-receiving sections 105a, 105b.

It is to be noted that the artery 3 is at a position generally symmetric to the light-emitting axis. The light-receiving efficiency is highest when the respective light-receiving sections receive the light equally. Therefore, it is desirable that the two light receiving sections should be placed at positions equally distant from the light-emitting axis, i.e., at symmetric positions.

Thus, providing two light-receiving sections at the positions symmetric to the light-emitting axis makes it possible to receive a light flux which cannot be collected by a single light-receiving section that the conventional ring sensor has, and therefore, the light-receiving efficiency can be enhanced. It goes without saying that providing not only two light-receiving sections but a number of light-receiving sections at the positions symmetric to the light-emitting axis also has the effect of enhancing the light-receiving efficiency. In other words, the light-receiving efficiency is enhanced by increasing the number of light-receiving sections and placing a plurality of the light-receiving sections at the positions symmetric to the optical axis of the light-emitting section.

According to an experiment, in the case where the first and second light receiving sections 105a, 105b were provided respectively at the positions of 90 degrees and 270 degrees, the proportion of the AC component in a light reception signal was large and pulse waves were sufficiently detected. In the case where they were provided at the positions of 135 degrees and 225 degrees, the proportion of the AC component was very small and detection of pulse waves was difficult.

The artery 3 is positioned closer to the front of the finger than the positions of 90 degrees and 270 degrees. Consequently, when the respective light-receiving sections are placed in the vicinity thereof, the reception signal receives pulses of the artery most, and therefore the proportion of the AC component increases.

Moreover, there is a bone on the back side of the finger, and the bone blocks outgoing light, the red light in particular, from the light emitting section 102. The bone is particularly thicker at the base of the finger than at the finger tip, and so this blocking is all the more notable.

In consideration of these conditions, the positions of the first and second light receiving sections 105a, 105b should preferably be the position from 90 degrees to 135 degrees and the position from 225 degrees to 270 degrees, respectively.

Moreover, since the light-receiving efficiency is enhanced, it becomes possible to proportionally reduce the light emission amount of the light-emitting section compared to the light emission amount of the conventional reflection-type ring sensor. In other words, by reducing and adjusting the light emission amount of the light-emitting section to the level that the signal-noise ratio of reception signals can sufficiently be obtained, stress on the skin such as low-temperature burn can be decreased, and the detector with safety in long-time wearing can be provided.

With this structure, providing two light-receiving section against one light-emitting section allows reduction in light emission amount and in driving current of the light-emitting section compared to the conventional structure having a single light-receiving section.

It is to be noted that in the present embodiment, an angle formed between the light-emitting axis and the light-receiving axis is generally rectangular, but the angle is not limited thereto.

Description is now given of a processing method for reception signals for achieving an optimum wearing position which maximizes the AC component in the reception signal in the present embodiment.

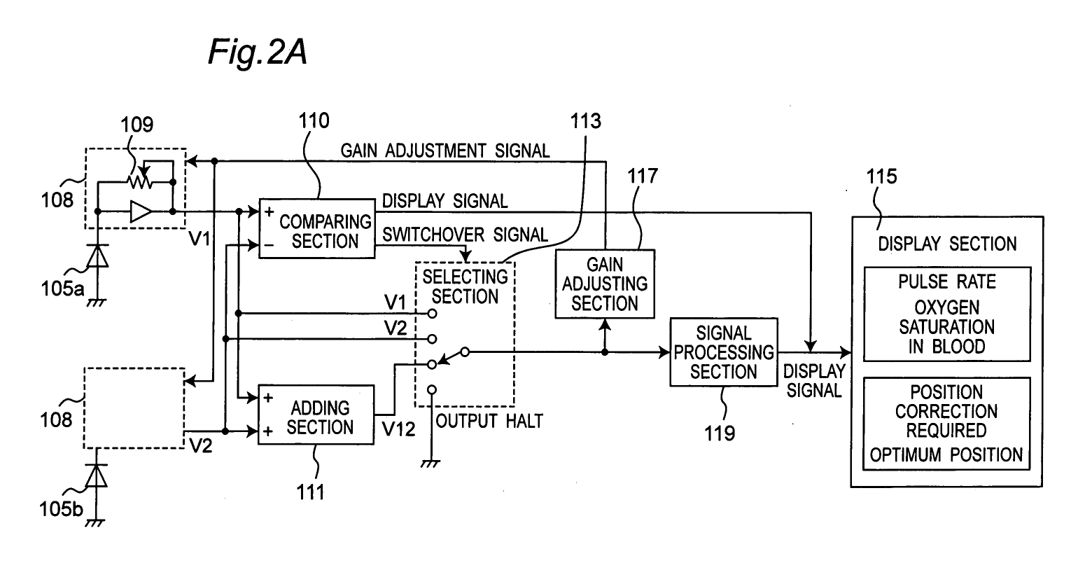

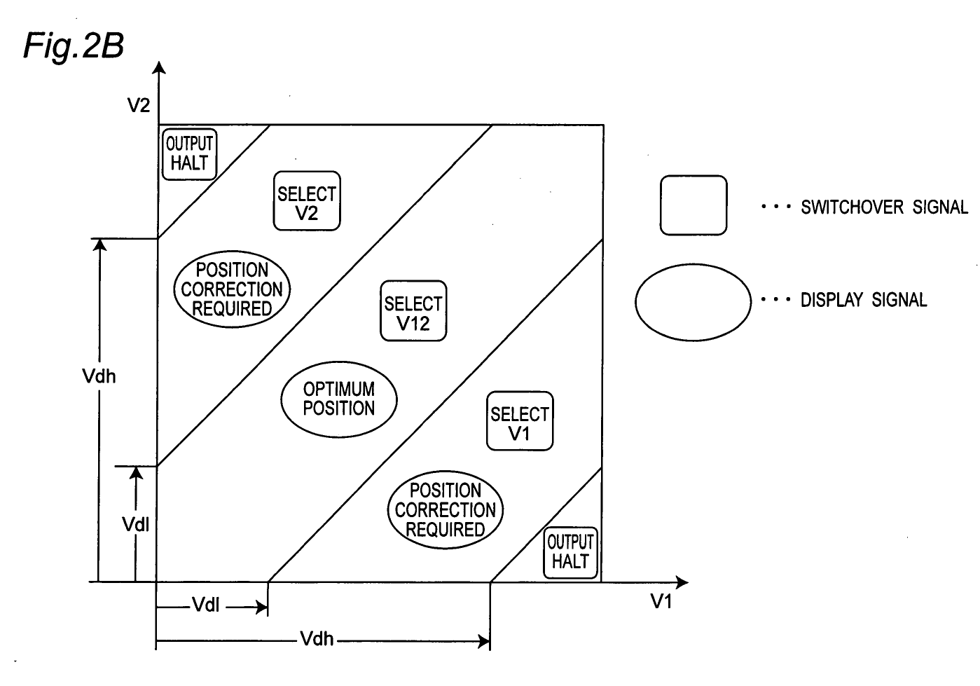

FIG. 2A is a block diagram showing a ring sensor, and FIG. 2B is a view showing the operation of a comparing section.

The first and second light receiving sections 105a, 105b output reception signals, which are photocurrents with intensity corresponding to the respective light reception amounts, to a IV (Current-Voltage) amplifier 108.

The IV amplifier 108 converts the photocurrents to voltages (hereinbelow referred to as pulse-wave voltages V1, V2) and output them to a comparing section 110 and to an adding section 111. The IV amplifier 108 can adjust gains with gain adjustment signals described below.

The comparing section 110 compares amplitudes of V1 and V2, and outputs a switchover signal to a selecting section 113 and a display signal to a display section 115 in response to the comparison result.

The adding section 111 outputs the sum of V1 and V2 (hereinbelow referred to as pulse-wave voltage V12).

The selecting section 113 selects any one of V1, V2, V12 and “output halt” based on the switchover signal, and outputs the result to a gain adjusting section 117 and to a signal processing section 119.

The signal processing section 119 calculates the output from the selecting section 113 to obtain biological information such as pulse rates and oxygen saturation in blood and outputs the obtained information to the display section 115.

The display section 115 displays biological information including a pulse rate, oxygen saturation in blood and a wearing position.

The gain adjusting section 117 detects a peak value of the pulse-wave voltage and outputs a gain adjustment signal corresponding to the peak value to the IV amplifier 108.

Description is now given of the operation of the comparing section 110, the selecting section 113 and the display section 115 with reference to FIG. 2B.

Specified values Vdl and Vdh are preset for identifying the light-receiving state of the light receiving sections 105 by comparison between V1 and V2 and for determining output values from the comparing section 110.

In the case of |V2−V1|≦Vdl, the selecting section 113 selects V12, and “optimum position” is displayed on the display section 115.

In the case of Vdl<V2−V1≦Vdh, the selecting section 113 selects V2, and in the case of Vdl<V1−V2≦Vdh, the selecting section 113 selects V1, and in both the cases, “position correction required” is displayed on the display section 115.

In the case of Vdh<|V2−V1|, output halt is selected.

Basically, if a difference of the pulse-wave voltages is within Vdl, then the sum of the pulse-wave voltages is selected. If a difference of the pulse-wave voltages is in the range of Vdl to Vdh, then the pulse-wave voltage with a larger amplitude is selected. If a difference of the pulse-wave voltages is beyond Vdh, then the wearing position is determined to be extremely displaced from the optimum position, and the output halt is selected to avoid detection of inaccurate pulse waves.

The artery 3 and the respective light-receiving sections are positioned generally symmetrically to the light-emitting axis. Consequently, even when the ring sensor 101 is displaced from the finger 1 in the rotation direction, one light-receiving section is positioned at the front of the finger, and therefore light reception is still possible. However, the other light-receiving section is positioned at the back of the finger, where the bone of the finger blocks light reception. Therefore, it is desirable to select the larger pulse-wave voltage by the selecting section 113.

It is to be noted that the values Vdl and Vdh are for identifying the light receiving state of the light receiving sections 105 as described above, and therefore appropriate values on Vdl and Vdh should be obtained by experiments so that a pulse wave signal can be attained in the most efficient way.

Thus, by providing two light-receiving sections and comparing the respective light reception signals, it becomes possible to display the state of the wearing position and to prompt the wearer to correct the wearing position. Moreover, automatic selection of the larger pulse-wave voltage by the selecting section can provide an optimum reception signal, and this allows stable detection of biological information.

It is to be noted that the information on the wearing position, “optimum position” and “position correction required”, may be color-coded with use of light-emitting devices having different luminous colors such as blue color and red color so as to draw attention of the wearer. Also, the display may be simplified so that only either “optimum position” or “position correction required” may be displayed to draw attention.

The gain adjusting section 117 is provided to avoid saturation of inputs into the comparing section 110, the adding section 111 and the signal processing section 119. The voltages V1 and V2 vary by body motion. In a certain state, the voltages exceed input capacities of the respective sections and cause problems such as distortion of signals. Gain adjustment is necessary to avoid these problems. More specifically, as shown in FIG. 2A, gain adjustment is achieved by adjusting values of feedback resistance 109 in the IV amplifier 108.

(Embodiment 2)

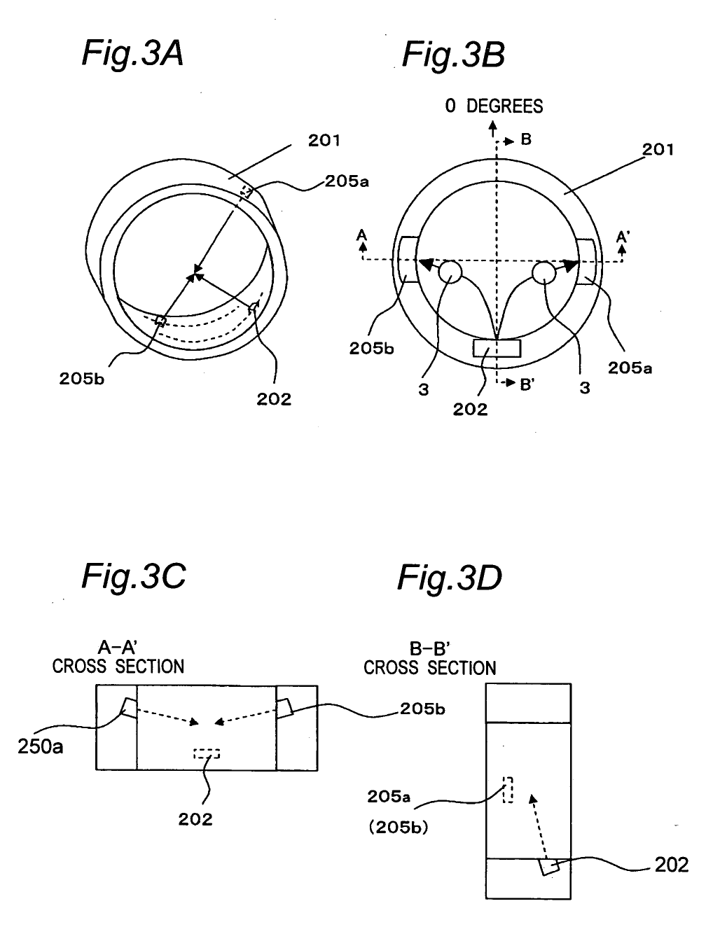

FIGS. 3A to 3D are views about a ring sensor in an embodiment 2.

It is to be noted that as for the structure of the embodiment 2, explanation about structure components identical to those in the embodiment 1 shown in FIGS. 1A to 1E and FIGS. 2A to 2B is omitted and their differences are mainly described.

In FIGS. 3A and 3B, a light-emitting section 202 is formed on the inner circumferential surface of a ring sensor 201, and first and second light-receiving sections 205a, 205b are formed at positions symmetric to a light-emitting axis of the light-emitting section 202. More specifically, the first and second light-receiving sections 205a, 205b are placed at positions facing each other on the inner circumferential surface of the ring sensor 201.

Moreover, a light-emitting point of the light-emitting section 202 and respective light-receiving points of the first and second light-receiving sections 205a, 205b are placed at tracks different from each other on the inner circumferential surface of the ring sensor 201 so that they are out of alignment and that the light-emitting axis and the light-receiving axes cross at a generally central point of the ring as shown in FIG. 3C.

In other words, if the ring is likened to a cylinder, light-receiving sections 205 would be provided near one opening while the light-emitting section 202 would be provided near the other opening, and the light-emitting axis would be inclined toward one opening side while the light-receiving axes would be inclined toward the other opening side.

Such placement makes it possible to suppress influence of disturbance light, in addition to the effect of the embodiment 1, since the light-emitting axis and the light-receiving axes are inclined toward the center of the ring.

It is to be noted that the method for processing reception signals for obtaining an optimum wearing position to maximize the AC component of the reception signals in the present embodiment is similar to that in the embodiment 1.

(Embodiment 3)

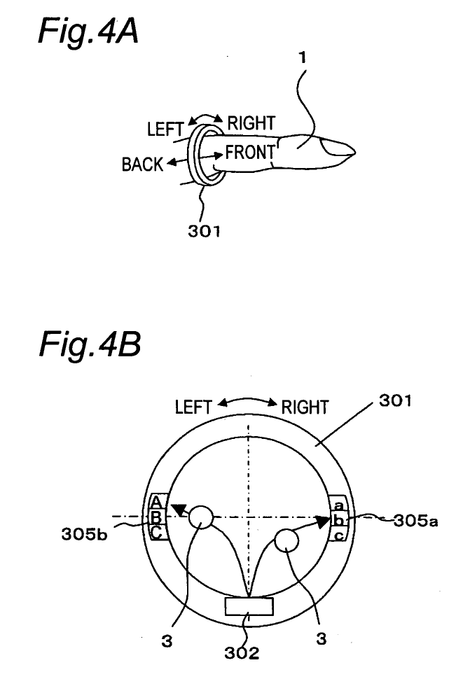

FIG. 4A is a perspective view showing a ring sensor in a worn state in an embodiment 3 of the present invention, and FIG. 4B is a transverse sectional view showing the ring sensor.

It is to be noted that as for the structure of the embodiment 3, explanation about structure components identical to those in the embodiments 1 and 2 shown in FIGS. 1A to 1E and FIGS. 3A to 3D is omitted and their differences are mainly described.

First and second light-receiving sections 305a, 305b are respectively divided into light-receiving regions a, b, c and A, B, C.

The respective light receiving regions are formed along the inner circumferential surface of the ring in a circumferential direction in the order of a, b, c and A, B, C from the farthest side from the a light-emitting section 302.

In FIG. 4B, the light-emitting section 302 is formed on the inner circumferential surface of a ring sensor 301, and the first and second light-receiving sections 305a, 305b are formed at positions symmetric to a light-emitting axis of the light-emitting section 302. More specifically, the first and second light-receiving sections 305a, 305b are placed at positions where the light-receiving regions b and B positioning at the central portions of the respective light receiving regions face each other on the inner circumferential surface of the ring sensor 301.

The light-emitting section 302 and the first and second light-receiving sections 305a, 305b may be placed so that the light-emitting axis and respective light-receiving axes are in the same plane as in the embodiment 1, or the light-emitting section 302 and the first and second light-receiving sections 305a, 305b may be placed so that a light-emitting point of the light-emitting section 302 and light-receiving points of the respective light receiving regions in the first and second light-receiving sections 305a, 305b are at different tracks on the inner circumferential surface of the ring sensor as in the embodiment 2.

Description is now given of a method for processing reception signals in the ring sensor having divided light receiving regions.

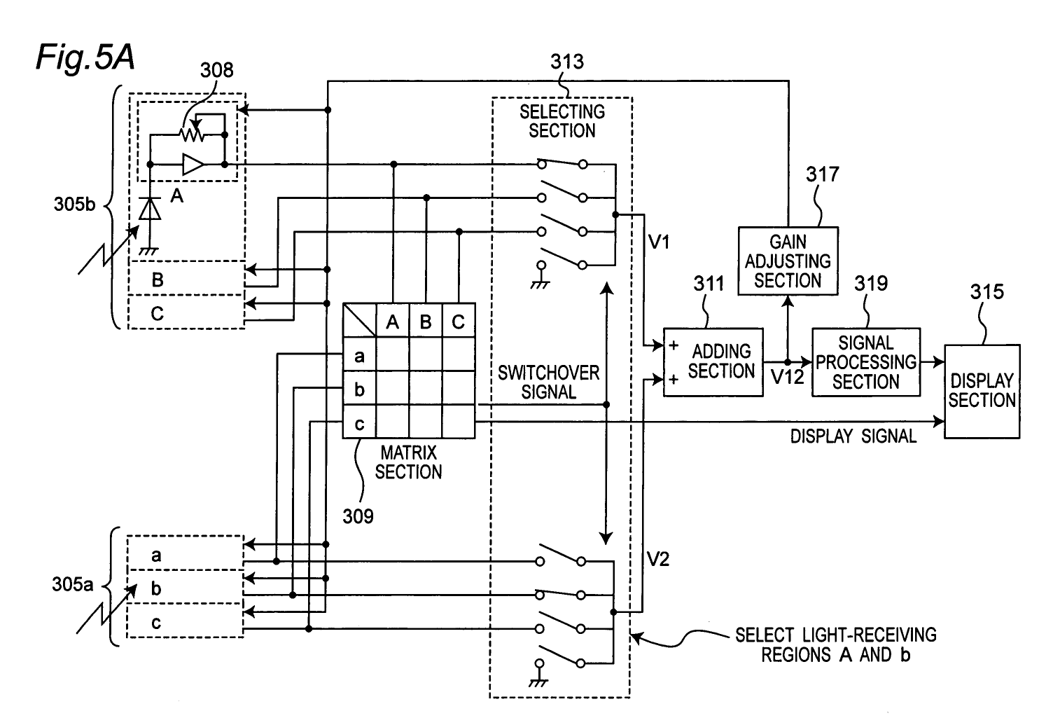

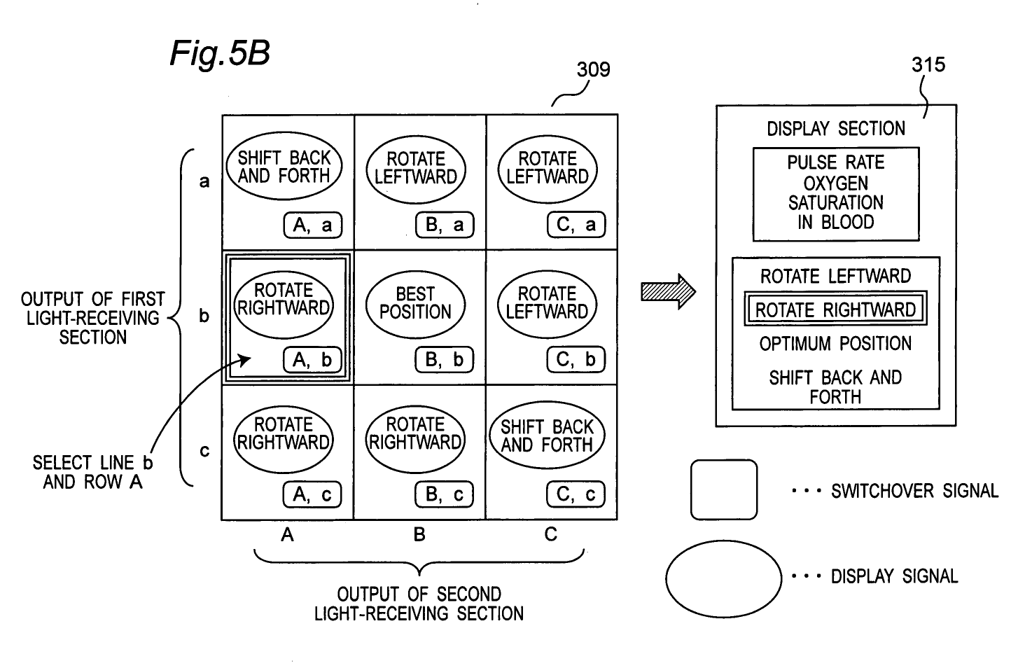

FIGS. 5A, 5B are a block diagram and a view showing a matrix and the operation of a display section of the ring sensor shown in FIGS. 4A, 4B.

Photocurrents corresponding to the respective light reception amounts in the respective light receiving regions in the respective light-receiving sections are converted to pulse-wave voltages by an IV amplifier 308 and outputted into a matrix section 309.

As described later, the matrix section 309 classifies light-receiving states of the light-receiving sections according to combinations of the light receiving regions which maximizes the amplitude of the pulse-wave voltages, and a switchover signal corresponding to the classification is outputted to a selecting section 313 and a display signal is outputted to a display section 315.

The selecting section 313 selects the pulse-wave voltages based on the switchover signal and outputs them to an adding section 311. The adding section 311 adds pulse-wave voltages V1 and V2 to obtain a sum V12, which is outputted into a gain adjusting section 317 and into a signal processing section 319.

Description is given of the operation of the matrix section 309, the selecting section 313 and the display section 315 with reference to FIG. 5B.

The matrix section 309 is composed of three lines from a to c and three rows from A to C, and the respective lines correspond to the light-receiving regions a to c while the respective rows correspond to the light-receiving regions A to C.

In each element of the matrix section 309, the switchover signal for determining a contact position of the selecting section 313 and the display signal for showing the correction direction of a ring wearing position are stored in advance.

When the respective light-receiving sections receive light, a combination of a line and a row corresponding to the light-receiving region with the highest pulse-wave voltage is first selected out of the regions a to c and A to C. Then, the switchover signal and the display signal stored in the element at a cross point between the selected line and row are outputted to the selecting section 313 and to the display section 315, respectively.

It is to be noted that the optimum wearing position of the ring is the position where the light receiving region b and B in the central portions of the respective light-receiving sections have the largest pulse-wave voltage.

The wearing position of the ring sensor may be displaced from the optimum position by body motion.

For example, in FIG. 4B, the light receiving regions b and A have the largest pulse-wave voltage. In this case, the ring is determined to be displaced from the optimum position in the leftward rotation direction, and therefore “rotate rightward” is displayed on the display section 315.

Thus, when the wearing position of the ring is displaced form the optimum position, the wearer is prompted to correct the wearing position.

Once the wearer corrects the wearing position to the optimum position by the display, the light receiving regions b and B are selected by the switchover signal, and “optimum position” is displayed on the display section 315.

When the wearing position is displaced again from the optimum position by body motion and the like, a switchover signal corresponding to the resultant matrix element is outputted, and a combination of the light receiving regions which maximizes the pulse-wave voltage is selected. The pulse-wave voltages of the respective light receiving regions are outputted into the adding section 311.

Thus, by dividing the light-receiving sections 305, the displacement direction of the ring can be detected. This brings about high convenience since the correction direction of the ring is displayed at the time of correcting the wearing position. This structure makes it possible, in addition to the effects of the embodiments 1 and 2, to enable the user to easily correct the wearing position, which allows stable detection of biological information.

Moreover, by setting the position where the light receiving regions b and B in the central portions of the respective light-receiving sections have the largest pulse-wave voltages as the optimum wearing position, the light receiving regions which maximize the pulse-wave voltages are selected in a swinging way with the light receiving regions b and B as a base point, and therefore stable pulse-wave signals can be obtained constantly. Moreover, the premise that the ring sensor 301 would be worn at the optimum position allows the structure in which V1 and V2 are added anytime.

It is also possible to structure the ring sensor so that as in the embodiment 1, the pulse-wave voltage with a larger amplitude is selected and if a difference between V1 and V2 exceeds a specified value, output halt is selected.

It is also possible to structure the ring sensor so that in the state of being worn at the optimum position, the light-receiving regions a to c and the light-receiving regions A to C, i.e., the entire light receiving regions, are selected.

It is to be noted that the first and second light-receiving sections 305a, 305b may be formed by a method for combining a photodiode having pre-divided light receiving regions and a lens and concentrating the light incident into each light-receiving section upon the respective light receiving regions with the lens. Without being limited to this, the first and second light-receiving sections 305a, 305b may be formed by a method for arraying a plurality of single light-receiving devices.

(Embodiment 4)

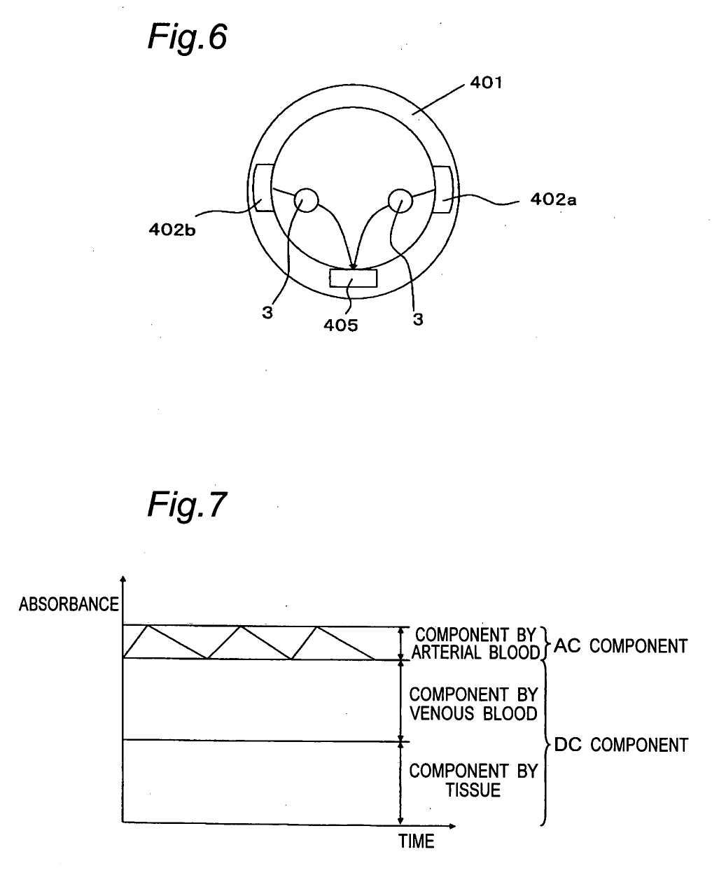

FIG. 6 is a transverse sectional view showing a ring sensor in an embodiment 4.

In FIG. 6, a light-receiving section 405 is formed on the inner circumferential surface of a ring sensor 401, and first and second light-emitting sections 402a, 402b are formed at positions symmetric to a light-receiving axis of the light-receiving section 405. The first and second light-emitting sections 402a, 402b face each other.

Light-emitting axes of the light-emitting sections 402a, 402b and a light-receiving axis of the light-receiving section 405 may be in the same plane. The light-emitting sections 402a, 402b and the light-receiving section 405 may also be formed so that respective light-emitting points of the light-emitting sections 402a, 402b and a light-receiving point of the light-receiving section 405 are at different tracks on the inner circumferential surface of the ring sensor 401 and that the light-emitting axes and the light-receiving axis cross at a generally central point of the ring.

In the thus-structured ring sensor 401, light emitted from the respective light-emitting sections 402a, 402b touch a blood vessel 3 of the finger and reflects or diffuses before reaching the light-receiving section 405.

Since the respective light-emitting sections 402a, 402b are placed at positions symmetric to the light-receiving axis, the light beams emitted from the respective light-emitting sections are equally applied to two arteries, and therefore pulse-waves can be detected equally.

Basically, the light amount necessary for detection of pulse waves, which has conventionally been attained from a single light-emitting section, is equally allotted to two light-emitting sections, so that the amount of outgoing light in one light-emitting section can be reduced.

Thus, by providing two light-emitting sections, the light emission amount in each light-emitting section can be reduced to generally half the light emission amount of the conventional ring sensor which emits light with a single light-emitting section, and still the same amount of light can be obtained.

Moreover, in the case where the ring sensor is worn for a long period of time, a light flux applied to a measurement target region per unit time is reduced, and therefore it becomes possible to decreases stress on the skin such as low-temperature burn and to ensure more safety.

An irradiation time of the same measurement target region can also be reduced to half by alternately lighting two light-emitting sections, and the same effect as above is also implemented.

Embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A detector, comprising:
a light-emitting section for radiating light; and
a light-receiving section for receiving light, the light-emitting section and the light-receiving section being formed on an inner circumferential surface of a finger ring-type ring,
wherein the light-receiving section is composed of at least a first light-receiving section and a second light-receiving section, and
wherein each light-receiving section is placed at a position symmetric to a light-emitting axis of the light-emitting section.

2. The detector according to claim 1, comprising:
a comparing section for comparing first and second reception signals outputted from the first and second light-receiving sections;
a selecting section for selecting any one of the first reception signal, the second reception signal, and a sum of the first and second reception signals based on a comparison result in the comparing section; and
a signal processing section for calculating biological information from the reception signal selected in the selecting section.

3. The detector according to claim 2,
wherein propriety of a wearing position is determined based on the comparison result.

4. The detector according to claim 1,
wherein the first and second light-receiving sections have a plurality of light-receiving regions along the inner circumferential surface.

5. The detector according to claim 4,
wherein the respective light-receiving sections have at least three light-receiving regions, and
wherein light-receiving regions each positioning in central portions of the respective light-receiving sections are placed at positions facing each other on the inner circumferential surface.

6. The detector according to claim 4,
wherein the first and second reception signals outputted from the first and second light-receiving sections are signals having a maximum amplitude among reception signals of the respective light-receiving regions, and propriety of a wearing position is determined by a combination of the light-receiving regions corresponding to the first and second reception signals.

7. The detector according to claim 5,
wherein the first and second reception signals outputted from the first and second light-receiving sections are signals having a maximum amplitude among reception signals of the respective light-receiving regions, and propriety of a wearing position is determined by a combination of the light-receiving regions corresponding to the first and second reception signals.

8. The detector according to claim 6 or 7,
wherein a correction direction of the wearing position is determined based on the combination.

9. The detector according to claim 6, comprising a signal processing section for calculating biological information from the reception signals selected based on the combination.

10. The detector according to claim 7, comprising a signal processing section for calculating biological information from the reception signals selected based on the combination.

11. The detector according to claim 1,
wherein the light-receiving axis of the light-receiving section and the light-emitting axis of the light-emitting section are in one plane.

12. The detector according to claim 1,
wherein a light-emitting point of the light-emitting section and a light-receiving point of the light-receiving section are placed at different tracks on the inner circumferential surface.

13. The detector according to claim 12,
wherein the light-emitting axis and the light-receiving axis are placed so as to intersect with each other.

14. The detector according to claim 1,
wherein the first light-receiving section and the second light-receiving section are placed at positions facing each other on the inner circumferential surface.

15. The detector according to claim 2, comprising an amplifier for amplifying a reception signal outputted from the light-receiving sections,
wherein an amplification factor of the amplifier is adjusted based on the reception signal inputted into the signal processing section.

16. The detector according to claim 2,
wherein the biological information calculated from the reception signal by the signal processing section is a pulse or oxygen saturation in blood.
